(19) United States
(12) Patent Application Publication
(10) Pub. No.: US 2009/0117184 A1
(43) Pub. Date: May 7, 2009

(54) Use of a gestagen in combination with an estrogen and one or more pharmaceutically acceptable auxiliary agents/excipients for lactose-free oral contraception

(76) Inventors: Sabine Fricke, Jena (DE); Manuela Pfeifer, Jena (DE); Claus Claussen, Jena (DE); Ralf Ladwig, Jena (DE); Beate Buerglen, Jena (DE)

Correspondence Address: Striker, Striker & Stenby, 103 East Neck Road, Huntington, NY, 11743, US

(21) Appl. No.: 12/258,817

(22) Filed: Oct. 27, 2008

Related U.S. Application Data
(60) Provisional application No. 60/985,443, filed on Nov. 5, 2007.

(51) Int. Cl. A61K 9/24; A61K 31/56; A61K 9/36

(52) U.S. Cl. 424/472; 514/170; 424/479

(57) ABSTRACT

Gestagens, preferably dienogest, chlormadinone acetate or levonorgestrel, in combination with estrogens, for example ethinylestradiol, 17β-estradiol or estradiol valerate, and one or more pharmaceutically acceptable auxiliary agents/excipients provide lactose-free oral contraception.

The possibility is provided of improving the prophylaxis for lactose intolerance concerning a possibly contributing factor and also in regard to the costly examinations for lactose intolerance.

The invention is also suitable for long-term use.

### TECHNICAL FIELD

The invention relates to the use of a gestagen in combination with an estrogen and one or more pharmaceutically acceptable auxiliary agents/excipients for producing a monophasic pharmaceutical preparation for lactose-free oral contraception. A possibility of improving the prophylaxis for lactose intolerance is provided concerning a possibly contributing factor and also as regards the expensive examinations for lactose intolerance.

### PRIOR ART

Lactose intolerance, also referred to as milk sugar intolerance, is a disease which in Germany occurs primarily in about 10 million people. Typical symptoms of lactose intolerance, depending on its severity, are abdominal pain, feeling of fullness, flatulence, nausea and diarrhea. Basically one differentiates between genetically caused lactose intolerance, for example one that is due to a congenital enzyme defect, or acquired lactose intolerance the cause of which is still not well known. Diagnosis of lactose intolerance requires a hydrogen breath test or a small-intestine biopsy. In the small intestine, lactose, a disaccharide and the hydrocarbon present in milk, is cleaved by lactase into the individual sugars which are absorbed by the mucous cells and carried further in the blood. If because of a lactase deficiency or reduced activity thereof the milk sugar or part of it reaches the large intestine uncleaved, it is here decomposed by the colonized bacteria into lactic and acetic acid, carbon dioxide, hydrogen and methane. As a result, an osmotic pressure is created causing increased intestinal peristalsis and diarrhea. At the same time, the gases formed in the intestine cause flatulence or cramps. Depending on the severity of the lactose intolerance, a lactose-free or low-lactose diet (<3 mg of lactose/day) is recommended to the affected person. A healthy adult ingests daily 20 to 30 g of lactose in a complete diet. 100 g of cow milk corresponds to about 5 g of lactose. Various kinds of bread and baked goods, sausages, butter and margarine, chocolate and sweetener tablets contain lactose for technological reasons.

Recommended therapy methods include lactase-containing enzyme preparations which are to be taken at the same time as food in order to stimulate milk sugar cleavage.

U.S. Pat. No. 6,881,428 discloses the preparation of lactose-free milk involving the addition of an enzyme (lactase) to the milk.

From the pharmaceutical technology it is known to prepare an oral contraceptive (OC) that is based exclusively on lactose. Lactose is especially well suited as formulation filler because of its outstanding properties. The resulting formulations are characterized by resistance, good decomposition properties and good stability. In a lactose granulate, the steroid hormones, alone or in combination, are very favorably distributed over the individual particle size classes. Demixing of the granulate, which would be indicated by an insufficiently uniform partition of the active ingredient content in the tablet cores, is here hardly known. It may be assumed that in a lactose-based oral contraceptive even very low doses of the active ingredient (at least 15 μg of ethinylestradiol per unit) would be uniformly distributed. Lactose itself can be readily granulated and the granulate can be processed to tablets without any difficulty. Direct tabletting which could conceivably be done with a special lactose has thus far, because of the nonuniformity of active ingredients in the individual formulations, not been taken into consideration and has not been used in practice. Until now, OC's with a low dose of active ingredients have been produced by granulation, followed by tabletting and in most cases by a coating process.

WO 2005030175 discloses a composition with norethisterone acetate and estradiol and cellulose binders and shows as an example a pharmaceutical composition containing up to 45% of lactose

WO 2005030176 discloses a composition with gestagens and cellulose binders and shows as an example a pharmaceutical composition containing besides norethisterone and estradiol also up to 45% of lactose.

### PRESENTATION OF THE INVENTION

The object of the invention is to provide an oral contraceptive for patients with lactose intolerance and for women who thus far have not yet recognized their lactose intolerance.

In this manner, the possibility is provided at the same time to improve the prophylaxis for lactose intolerance concerning a possibly contributing factor and also in regard to the costly examinations of lactose intolerance.

We have now found that according to the invention this objective is reached by use of a combination of estrogens and one or more pharmaceutically acceptable auxiliary agents/excipients for producing a pharmaceutical preparation for lactose-free oral contraception. Preferably, the gestagen used is dienogest at a dose of 2.0 mg or 1.5 mg or chlormadinone acetate or levonorgestrel at an equivalent dosage, and the estrogen used is ethinylestradiol at a dose of 0.030 mg or 0.020 mg or 0.015 mg or estradiol or estradiol valerate at an equivalent dosage. The ethinylestradiol can also be used in the form of a clathrate.

Other gestagens can conceivably also be used for the purpose of the invention.

The preparation of the lactose-free oral contraceptives of the invention is carried out using cellulose as a base material. The formulations are made by granulation and tabletting and often also by coating.

A method of preparation of low-dose OC's that thus far has not been practiced is the use of cellullose as filler and the granulation of the active ingredients with binders. Preferred binders are hydroxypropylcellulose (HPC) used in an amount of 1 to 5% based on the weight of the core. Hypromellose and maltodextrin or gelatin or starch paste, however, can also be used as binders. This has thus far not been taken into consideration, primarily because of problems of active ingredient distribution caused by the fact that celluloses have a very limited particle size distribution.

According to the invention, the objective was also reached in that by selection and optionally combination of different kinds of microcrystalline cellulose (MCC) that differ in bulk density, particle size and moisture content, for example, Avicel PH 101 or Avicel PH 102 or Avicel PH 112 or combinations of MCC and dibasic calcium phosphate or mannitol with at the same time an optimum selection of the binder-to-total weight ratio of the formulation (1-5% to 100%), we have, surprisingly, achieved uniform partition of the mostly very low-dosed gestagens and estrogens in the individual granulate fractions and, hence, also in the tablet. For better distribution in the case of the particularly low dosed EE (15 μg per tablet is currently possible as a minimum), the active ingredient is sprayed into the fluidized bed during the granulation process as an ethanolic ethinylestradiol solution.

The objective is also reached by use of the process for producing a monophasic pharmaceutical preparation for lactose-free oral contraception in which a combination of a gestagen and an estrogen is used in n×21 daily dose units followed by at the most 7 daily hormone-free or placebo-containing dose units and wherein n is equal to 2, 3, 4, 5, 6, 7, 8, 9, 10, 11, 12, 13, 14, 15, 16 and 17. Advantageous embodiments of the invention are indicated in claims 18 to 27.

### PRACTICAL EXAMPLES

### Example 1

### Active Ingredient Combination

2.0 mg of dienogest (DNG)/0.030 mg of ethinylestradiol (EE)

### Example 2

### Active Ingredient Combination

2.0 mg of chlormadinone acetate (CMA)/0.030 mg of EE

### Example 3

### Active Ingredient Combination

0.125 mg of levonorgestrel (LNG)/0.030 mg of EE

### Example 4

1.5 mg of DNG/0.015 mg of EE of which 0.825 mg of DNG and 0.015 mg of EE are film constituents

The detailed composition of the practical examples is described in Table 1.

Table 2 shows the comparison of various cellulose formulations in terms of active ingredient distribution in the granulate and tablet cores as a function of the type of cellulose and quantity of binder.

Uniform distribution of the active ingredients is indicated by determining the active ingredient contents in the individual screen fractions (fine, medium, coarse) and by determining the CUT of the tablet cores during tabletting (beginning, middle, end).

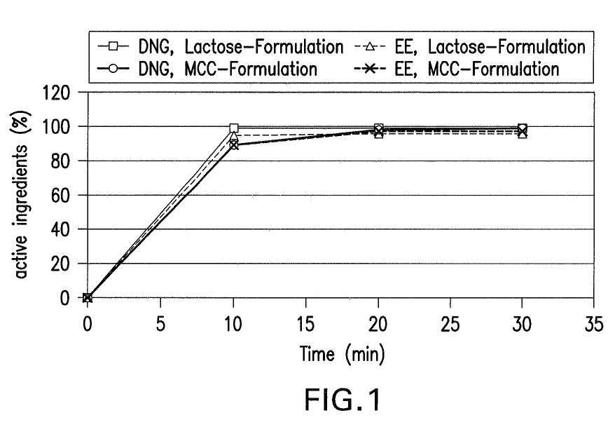

FIG. 1 shows the release diagrams of the cellulose-containing combination (2.0 mg of DNG and 0.30 mg of EE) in comparison with the lactose-containing combination (2.0 mg of DNG and 0.030 mg of EE) as determined by the dissolution test in the release apparatus: paddle agitator and using 37° C. water as dissolution medium at a rotation rate of 100 rpm. As can be seen, release from the lactose-free, cellulose-containing active ingredient is the same as from the lactose-containing active ingredient combination.

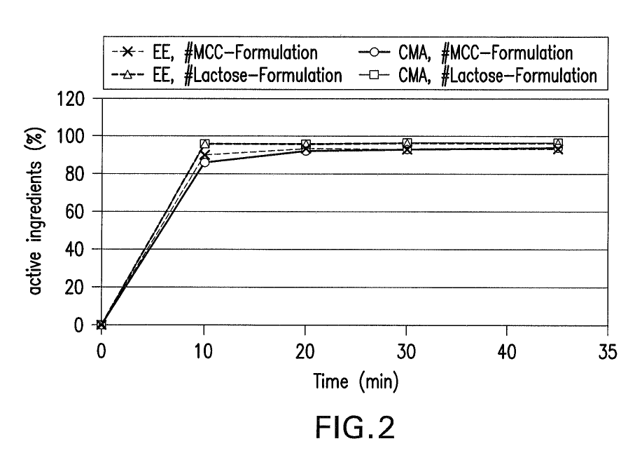

FIG. 2 shows the release diagrams of the cellulose-containing combination (2.0 mg of CMA and 0.30 mg of EE) in comparison with the lactose-containing combination (2.0 mg of CMA and 0.030 mg of EE) as determined by the dissolution test in the release apparatus: paddle agitator and using sodium dodecylsulfate at 37° C. as dissolution medium and a rotation rate of 75 rpm. As can be seen, release from the lactose-free, cellulose-containing active ingredient combination is the same as from the cellulose-containing active ingredient combination.

TABLE 1

| Formulation | | | | |
|---|---|---|---|---|
| (Core) | 1 | 2 | 3 | 4 (MR)* |
| Active | 2 mg of DNG | 2 mg of CMA | 0.125 mg of | 0.675 mg |
| ingredient | 0.03 mg of EE | 0.03 mg of EE | LNG | of DNG |
| | | | 0.03 mg of EE | |
| Filler | 43.52 mg | 43.52 mg | 45.395 mg | 58.425 mg |
| | of MCC | of MCC | of MCC | of MCC |
| Matrix former | — | — | — | 9 mg of hypromellose |
| Disintegrant | 1.95 mg of | 1.95 mg of | 1.95 mg of | 15 mg of corn |
| | croscarmellose | croscarmellose | croscarmellose | starch |
| | sodium | sodium | sodium | |
| Binder | 2 mg of | 2 mg of hydroxy- | 2 mg of | 6 mg of maltodextrin |
| | hydroxy- | propyl- | hydroxy- | |
| | propyl- | cellulose | propyl- | |
| | cellulose | | cellulose | |
| Lubricant | 0.5 mg of | 0.5 mg of magnesium | 0.5 mg of magnesium | 0.9 mg of magnesium |
| | magnesium | stearate | stearate | stearate |
| | stearate | | | |

*0.825 mg of DNG and 0.015 mg of EE are used additionally as film constituents in an active ingredient layer.

TABLE 2

| | | Lot No. 550907 | Lot No. 621007 | Lot No. 631007 |
|---|---|---|---|---|
| Composition | | CMA 2 mg | CMA 2 mg | CMA 2 mg |
| | | EE 0.03 mg | EE 0.03 mg | EE 0.03 mg |
| | | Avicel PH 102 | Avicel PH 102 | Avicel PH 101 |
| | | 44.52 mg | 28.52 mg | 36.02 mg |
| | | HPC 1 mg | Avicel PH 112 | Avicel PH 112 |
| | | croscarmellose Na | 15 mg | 7.5 mg |
| | | 1.95 mg | HPC 2 mg | HPC 2 mg |
| | | Mg stearate 0.5 mg | croscarmellose Na | Croscarmellose Na |
| | | | 1.95 mg | 1.95 mg |
| | | | Mg stearate 0.5 mg | Mg Stearate 0.5 mg |
| Distribution | Fine | CMA 125% | CMA 90.5% | CMA 99.4% |
| Screen Fractions | | EE 148% | EE 104.9% | EE 107.7% |
| | Medium | CMA 92% | CMA 114.9% | CMA 97.3% |
| | | EE 93% | EE 118.5% | EE 96.9% |
| | Coarse | CMA 79% | CMA 125.9% | CMA 111.9% |
| | | EE 52% | EE 116% | EE 102.5% |
| CUT Cores | Start | CMA 2.75 | CMA 3.89 | CMA 2.27 |
| (AV Value) | | EE 3.78 | EE 4.20 | EE 9.65 |
| Std.: <15 | Middle | CMA 4.59 | CMA 7.15 | CMA 3.74 |
| | | EE 5.33 | EE 8.59 | EE 3.01 |
| | End | CMA 4.68 | CMA 3.58 | CMA 7.12 |
| | | EE 4.62 | EE 6.71 | EE 9.24 |

1. Use of a gestagen in combination with an estrogen and one or more pharmaceutically acceptable auxiliary agents/excipients for producing a monophasic pharmaceutical preparation for lactose-free oral contraception.

2. Use as defined in claim 1, characterized in that the gestagen component is 17α-cyanomethyl-17-β-hydroxyestra-4,9-dien-3-one (dienogest), chlormadinone acetate or levonorgestrel.

3. Use as defined in claim 1, characterized in that the estrogen component is a synthetic estrogen or a natural estrogen or an ester thereof.

4. Use as defined in claim 1, characterized in that the synthetic estrogen is ethinylestradiol and the natural estrogen is 17β-estradiol (estradiol) or estradiol valerate.

5. Use as defined in claim 1, characterized in that the daily gestagen dose is equal to or less than 2 mg of dienogest or an equivalent amount of chlormadinone acetate or levonorgestrel.

6. Use as defined in claim 1, characterized in that the daily gestagen dose amounts to 2 mg or 1.5 mg of dienogest or an equivalent amount of chlormadinone acetate or levonorgestrel.

7. Use as defined in claim 1, characterized in that the daily estrogen dose is equal to or less than 0.030 mg of ethinylestradiol or an equivalent amount of estradiol or estradiol valerate.

8. Use as defined in claim 1, characterized in that the daily estrogen dose amounts to 0.030 mg or 0.020 mg or 0.015 mg of ethinylestradiol or an equivalent amount of estradiol or estradiol valerate.

9. Use as defined in claim 1, characterized in that the pharmaceutical preparation is produced for the use of at least 21 daily dose units and at the most 7 hormone-free or placebo-containing dose units of the pharmaceutical preparation with one or more pharmaceutically acceptable auxiliary agents/excipients.

10. Use as defined in claim 1, characterized in that the pharmaceutical preparation is produced for the use of 21, 22, 23, 24 or 25 daily dose units of the pharmaceutical preparation and of 7, 6, 5, 4 or 3 hormone-free or placebo-containing daily dose units so that the total number of cycle days is 28.

11. Use as defined in claim 1, characterized in that the pharmaceutical preparation is produced for the use of at least n×21 daily dose units of the pharmaceutical preparation where n equals 2, 3, 4, 5, 6, 7, 8, 9, 10, 11, 12, 13, 14, 15, 16 and 17 and of a maximum of 7 daily hormone-free or placebo-containing dose units.

12. Use as defined in claim 11, characterized in that the pharmaceutical preparation is produced for the use of 3, 4, 5, 6 or 7 daily hormone-free or placebo-containing dose units.

13. Use as defined in claim 12, characterized in that the pharmaceutical preparation is produced for the use of 84 daily dose units with the combination of gestagen and estrogen and of 7 hormone-free or placebo-containing daily dose units so that the total number of cycle days per year is 4×(n×21 plus 7) where n equals 4.

14. Use as defined in claim 1, characterized in that the pharmaceutical preparation is in the form of tablets, film tablets, sugar-coated tablets, capsules or powder.

15. Use as defined in claim 1, characterized in that the drug form is a film tablet consisting of a tablet core containing part of the total amount of gestagen that is to be released in retarded manner and a film coating containing part of the total amount of the gestagen that is to be released in a non-retarded manner (fast) and the total amount of the estrogen that is to be released in a non-retarded manner (fast).

16. Use as defined in claim 15, characterized in that at least 10% and preferably 30% of the gestagen is dissolved out of the tablet core in retarded manner after more than 30 minutes as determined by the dissolution test using 37° C. water as the dissolution medium at a rotation rate of 50 rpm.

17. Use as defined in claim 15, characterized in that the gestagen is dienogest or chlormadinone acetate and the estrogen is ethinylestradiol.

18. Process for producing a monophasic pharmaceutical preparation for lactose-free oral contraception, characterized in that a combination of a gestagen and an estrogen is used in n×21 daily dose units followed by at the most 7 daily hormone-free or placebo-containing dose units where n equals 2, 3, 4, 5, 6, 7, 8, 9, 10, 11, 12, 13, 14, 15, 16 and 17.

19. Process as defined in claim 18, characterized in that the gestagen component is 17α-cyanomethyl-17-β-hydroxyestra-4,9-dien-3-one (dienogest), chlormadinone acetate or levonorgestrel.

20. Process as defined in claim 18, characterized in that the estrogen component is a synthetic estrogen or a natural estrogen or an ester thereof.

21. Process as defined in claim 18, characterized in that the synthetic estrogen is ethinylestradiol and the natural estrogen is 17β-estradiol (estradiol) or estradiol valerate.

22. Process as defined in claim 18, characterized in that the daily gestagen dose is equal to or less than 2 mg of dienogest or an equivalent amount of chlormadinone acetate or levonorgestrel.

23. Process as defined in claim 18, characterized in that the daily gestagen dose amounts to 2 mg or 1.5 mg of dienogest or an equivalent amount of chlormadinone acetate or levonorgestrel.

24. Process as defined in claim 1, characterized in that the daily estrogen dose is equal to or less than 0.030 mg of ethinylestradiol or an equivalent amount of estradiol or estradiol valerate.

25. Process as defined in claim 1, characterized in that the daily dose of estrogen is 0.030 mg or 0.020 mg or 0.015 mg of ethinylestradiol or an equivalent amount of estradiol or estradiol valerate.

26. Process as defined in claim 1, characterized in that the drug form of the monophasic pharmaceutical preparation is a film tablet consisting of a tablet core containing part of the total dienogest or chlormadinone acetate or levonorgestrel that is to be released in retarded manner and a film coating containing part of the total amount of dienogest or chlormadinone acetate or levonorgestrel that is to be released in non-retarded manner (fast) and the total amount of ethinylestradiol or estradiol valerate that is to be released in non-retarded manner (fast).

27. Process as defined in claim 1, characterized in that at least 10% and preferably 30% of the dienogest is dissolved out of the tablet core after more than 30 minutes as determined by the dissolution test using 37° C. water as the dissolution medium at a rotation rate of 50 rpm.
